(19) United States
(12) Patent Application Publication
(10) Pub. No.: US 2008/0254011 A1
(43) Pub. Date: Oct. 16, 2008

(54) Use of selected lactic acid bacteria for reducing atherosclerosis

(76) Inventors: Peter Rothschild, Lidingo (SE); Eamonn Connolly, Lidingo (SE); Bo Mollstam, Lerum (SE)

Correspondence Address: LYNN E BARBER, P O BOX 16528, FORT WORTH, TX, 76162, US

(21) Appl. No.: 11/786,356

(22) Filed: Apr. 11, 2007

(51) Int. Cl. A61K 35/74; A23C 9/12; A61P 9/10; C12N 1/20

(52) U.S. Cl. 424/93.45; 426/61; 435/252.1

(57) ABSTRACT

Strains of lactic acid bacteria selected for their capability of increasing the BSH-activity and consequently lowering serum LDL-cholesterol, and simultaneously decreasing the pro-inflammatory cytokine TNF-α levels, for prophylaxis and/or treatment of atherosclerosis and other cardiovascular diseases, a method of selecting such strains, and products containing such strains.

### FIELD OF THE INVENTION

The invention herein provides certain strains of lactic acid bacteria selected for their capability of increasing the activity of bile salt hydrolase (BSH) and consequently lowering serum LDL-cholesterol, and simultaneously decreasing the pro-inflammatory cytokine Tumor Necrosis Factor-α (TNF-α) levels, for prophylaxis and/or treatment of atherosclerosis and other cardiovascular diseases, a method of selecting such strains, and products containing such strains.

### BACKGROUND OF THE INVENTION

Probiotics

Probiotics have been shown to have beneficial health effects (Gorbach, S. L. 2000. Probiotics and gastrointestinal health. Am. J. Gastroenterol. 95:S2-S4). Many different activities have been ascribed to probiotics; however, the mechanisms whereby these effects are achieved are poorly understood. The effects include enhanced innate and acquired immunity (Gill, H. S., K. J. Rutherfurd, J. Prasad, and P. K Gopal. 2000. Enhancement of natural and acquired immunity by Lactobacillus rhamnosus (HN001), Lactobacillus acidophilus (HN017) and Bifidobacterium lactis (HN019). Br. J. Nutr. 83:167-176), increased anti-inflammatory cytokine production (IL-10) (Pessi, T., Y. Sutas, M. Hurme, and E. Isolauri. 2000. Interleukin-10 generation in atopic children following oral Lactobacillus rhamnosus GG. Clin. Exp. Allergy 30:1804-1808), and reduced intestinal permeability (Madsen, K., A. Cornish, P. Soper, C. McKaigney, H. Jijon, C. Yachimec, J. Doyle, L. Jewell, and C. De Simone. 2001. Probiotic bacteria enhance marine and human intestinal epithelial barrier function. Gastroenterology 121:580-591). Various strains of Lactobacillus have been particularly well studied both in animals and humans. They may be effective in preventing and treating traveler's diarrhea (Marteau, P. R., M. de Vrese, C. J. Cellier, and J. Schrezenmeir. 2001. Protection from gastrointestinal diseases with the use of probiotics. Am. J. Clin. Nutr. 73:430 S-436S), recurrent Clostridium difficile infection (Gorbach, S. L. 1987. Bacterial diarrhoea and its treatment. Lancet ii:1378-1382), rotavirus (Szajewska, H., M. Kotowska, J. Z. Mrukowicz, M. Armanska, and W. Mikolajczyk. 2001. Efficacy of Lactobacillus GG (L.GG) in prevention of nosocomial diarrhea in infants. J. Pediatr. 138:361-365), and Helicobacter infections (Mukai, T., T. Asasaka, E. Sato, K. Mori, M. Matsumoto, and H Ohori. 2002. Inhibition of binding of Helicobacter pylori to the glycolipid receptors by probiotic Lactobacillus reuteri. FEMS Immunol. Med. Microbiol. 32:105-110). L. Reuteri isolated from mouse intestine inhibited the onset of colitis in IL-10 transgenic knockout mice (Madsen, K. L., J. S. Doyle, L. D. Jewell, M. M. Tavernini, and R. N. Fedorak. 1999. Lactobacillus species prevents colitis in interleukin 10 gene-deficient mice. Gastroenterology 116:1107-1114). A clinical trial with a mixture of probiotics has shown significant improvement in chronic pouchitis (Gionchetti, P., F. Rizzello, A. Venturi, P. Brigidi, D. Matteuzzi, G. Bazzocchi, G. Poggioli, M. Miglioli, and M. Campieri. 2000. Oral bacteriotherapy as maintenance treatment in patients with chronic pouchitis: a double-blind, placebo-controlled trial. Gastroenterology 119:305-309).

Immune Responses (Th-1/Th2/TR)

Inflammation is mediated by intercellular signal proteins known as cytokines, which are produced by macrophages and dendritic cells in the epithelium in response to an antigenic stimulus. Upon contact between the epithelium and the antigen, antigen presenting cells (including dendritic cells) in the epithelium propagate the signal to naive macrophages which then respond in a so-called Th-1 type response in which pro-inflammatory cytokines including TNFα, IL-1, IL-6, IL-12 are produced by the macrophages. These cytokines in turn stimulate natural killer cells, T-cells and other cells to produce interferon γ (IFNγ), which is the key mediator of inflammation. Naive macrophages can also respond to antigens with a Th-2 type response. This response is suppressed by IFNγ. These Th-2 type cells produce anti-inflammatory cytokines such as IL-4, IL-5, IL-9 and IL-10.

IL-10 is known to inhibit the production of IFNγ and thus dampen the immune response. The balance between Th-1 and Th-2 type cells and their respective cytokine production defines the extent of the inflammation response to a given antigen. Th-2 type cells can also stimulate the production of immunoglobulins via the immune system. Anti-inflammatory activity in the gastrointestinal tract, where there is a reduced TNFα level, correlates with enhanced epithelial cells (gut wall lining integrity) and thus to a reduction in the negative effects caused by gastrointestinal pathogens and toxins.

T-regulatory (TR) cells are viewed as an integral component of the immune response. These cells primarily appear to fine-tune protective antimicrobial immunity in order to minimize harmful immune pathology (Powrie F, Maloy K J. 2003. Regulating the regulators, Science 299 1030-1031). TR cells were shown to produce increased levels of the anti-inflammatory cytokine IL-10 (Smits, H. H., A. Engering, D. van der Kleij, E. C. de Jong, K. Schipper, T. M. van Capel, B. A. J. Zaat, M. Yazdanbakhsh, E. A. Wierenga, Y. van Kooyk, and L. Kapsenberg. 2005. Selective probiotic bacteria induce IL-10-producing regulatory T cells in vitro by modulating dendritic cell function through dendritic cell-specific intercellular adhesion molecule 3-grabbing nonintegrin. J Allergy Clin Immunol. 115:1260-1267). Factors controlling the development and activation of TR cells should enable shifting of the equilibrium either toward TR cell activity (to treat autoimmune diseases and to enhance survival of organ transplants), or away from TR cell activity (to boost vaccination and tumor rejection)(Walter J. Dobrogosz. Enhancement of human health with L. reuteri, A Probiotic, Immunobiotic and Immunoprobiotic. NUTRAfoods: 2005: 4(⅔) 15-28).

Immunomodulatory Effects of Probiotics

Lactobacillus rhamnosus strain GG (LGG) is a potential probiotic agent, with multiple studies having demonstrated the ability of LGG to colonize the intestinal tract and modulate mucosal epithelial and immune responses. LGG increased enterocyte proliferation and villous size in mono-associated gnotobiotic rats (Banasaz, M., E. Norin, R. Holma, and T. Midtvedt. 2002. Increased enterocyte production in gnotobiotic rats mono-associated with Lactobacillus rhamnosus GG. Appl Environ Microbiol. 68: 3031-3034). LGG also modulates the proliferation of murine lymphocyte responses ex vivo following oral administration (Kirjavainen, P. V., H. S. ElNezami, S. J; Salminen, J. T. Ahokas, and P. F. Wright. 1999. Effects of orally administered viable Lactobacillus rhamnosus GG and Propionibacterium freudenreichii subsp. shermanii JS on mouse lymphocyte proliferation. Clin Diagn Lab Immunol 6: 799-802) and L. paracasei alters modulatory cytokine profiles of CD4+ T lymphocytes (Von der Weid T., C. Bulliard, and E. J. Schiffrin. 2001. Induction by a lactic acid bacterium of a population of CD4(+) T cells with low proliferative capacity that produce transforming growth factor beta and interleukin-10. Clin Diagn Lab Immunol 8: 695-701). In addition to adaptive immune responses, LGG has effects on innate immune responses. LGG activates nuclear factor kappa B (NF-κB) and signal transducer and activator of transcription (STAT) signaling pathways in human macrophages (Miettinen, M., A. Lehtonen, I. Julkunen, and S. Matikainen. 2000. Lactobacilli and Streptococci activate NF-kappa B and STAT signaling pathways in human macrophages. J Immunol 164: 3733-3740), and L. rhamnosus stimulates interleukin-12 (IL-12) production by macrophages (Hessle, C., L. A. Hanson, and A. E. Wold. 1999. Lactobacilli from human gastrointestinal mucosa are strong stimulators of IL-12 production. Clin Exp Immunol 116: 276-282). LGG also stimulates production of immunomodulatory cytokines such as IL-10 in children (Pessi, T., Y. Sutas, M. Hurme, and E. Isolauri. 2000. Interleukin-10 generation in atopic children following oral Lactobacillus rhamnosus GG. Clin Exp Allergy 30: 1804-1808) and may regulate pro-inflammatory responses in vivo. Effector cells of innate immunity, such as macrophages, dendritic cells and neutrophils, are the primary drivers for the majority of inflammatory responses (Janeway, C. A., Jr. and R. Medzhitov. 2002. Innate immune recognition. Annu Rev Immunol 20: 197-216). The thought that innate immunity dictates the course of both innate and adaptive responses to antigens as self or non-self emphasizes the role of the innate immunity in controlling inflammation.

U.S. Patent Application No. 20020019043 relates to treating inflammatory bowel disease by administering a cytokine-producing Gram-positive bacteria or a cytokine antagonist-producing Gram-positive bacterial strain. In specific embodiments, the cytokine or cytokine antagonist are selected from IL-10, a soluble TNF-α receptor or another TNF-α antagonist, an IL-12 antagonist, an interferon-gamma antagonist, an IL-1 antagonist, and others. In specific embodiments, the Gram-positive bacteria are genetically engineered to produce a cytokine, cytokine antagonist, and so forth.

Immunomodulatory Effects of L. reuteri

Immunomodulatory effects of L. reuteri was for example reported by Christensen who showed that probiotic lactobacilli exerted their immunomodulatory effects by modulating the Th1/Th2/Th3/Tr1/TR-promoting capacity of dendritic cells (DCs) (Christensen H. R., H. Frokiaer, and J. J. Pestka. 2002. Lactobacilli differentially modulate expression of cytokines and maturation surface markers in murine dendritic cells. J. Immunol. 168: 171-178). They showed that when murine DCs were exposed to co-cultures of different Lactobacillus strains, including L. reuteri strains, they were differentially modulated for production of cytokines 11-6, IL-10, IL-12, and TNF-a, and for up-regulation of MHC class II and CD86 surface markers in a concentration dependent manner. All lactobacilli upregulated surface MHC class. 11 and CD86 markers—indicative of DC maturation. Particularly notable in these studies was that L. reuteri (strain 12246) was a poor IL-12 inducer, but when in co-culture with L. johnsonii or L. casei, it differentially inhibited production of the pro-inflammatory cytokine signals IL-12, IL-6 and TNF-α which were stimulated by the latter two species. IL-10 production remained unaltered under these conditions. These findings led to their conclusions that ‘L. reuteri may contribute to an environmental modulation of the intestinal dendritic cell generation favoring tolerance toward antigens bearing no ‘danger signal’ while at the same time keeping intact the capacity to respond against pathogens recognized via a danger signal like LPS.’ They also concluded that some strains of L. reuteri might be a potential fine-targeted treatment effective for down-regulating production of IL-12 and TNF-α (and IL-6) while inducing the anti-inflammatory IL-10, thus representing an alternative therapeutic approach to counterbalance the pro-inflammatory intestinal cytokine milieu.

Smits extended these observations and showed that L. reuteri has the ability to prime DCs to stimulate T regulatory (TR) cell production. They used three different Lactobacillus species co-cultured in vitro with human monocyte-derived DCs. Two of the lactobacilli, a human L. reuteri strain (ATCC 53609) and L. casei, but not an L. plantarum strain, primed these DCs to stimulate development of TR cells. These TR cells were shown to produce increased levels of IL-10 and were able to inhibit proliferation of bystander T cells in an IL-10-dependent fashion (Smits, H. H., A. Engering, D. van der Kleij, E. C. de Jong, K. Schipper, T. M. M. van Capel B. A. J. Zaat, M. Yazdanbakhsh, E. A. Wierenga, Y. van Kooyk, and L. Kapsenberg. 2005. Selective probiotic bacteria induce IL-10-producing regulatory T cells in vitro by modulating dendritic cell function through dendritic cell-specific intercellular adhesion molecule 3-grabbing nonintegrin. J Allergy Clin Immunol. 115:1260-1267). These studies on L. reuteri-DC interactions are viewed in connection with ground-breaking studies by Hori (Hori S, T. Nomura, and S. Sakaguchi. 2003. Control of regulatory T cell development by the transcription factor Foxp3. Science. 299:1057-1061) and Pasare and Medzhitov (Pasare C. and R. Medzhitov. 2003. Toll pathway-dependent blockade of CD4+ Cd25+T cell-mediated suppression by dendritic cells. Science 299:1033-1036) has provided valuable insights into one of L. reuteri's immunobiotic modes of action.

Nerve growth factor (NGF), in addition to its activity on neuronal cell growth, has significant, anti-inflammatory effects in several experimental systems in vitro and in vivo, including a model of colitis. Ma et al. (2004) explored the mechanism of effect of L. reuteri in the human epithelial cell lines on cytokine and NGF synthesis and IL-8 response to TNF-α. They concluded that L. reuteri has potent direct anti-inflammatory activity on human epithelial cells, which is likely to be related to the activity of ingested probiotics. They also concluded that L. reuteri upregulates the unusual anti-inflammatory molecule, NGF, and inhibits NF-κB translocation to the nucleus (Ma, D., P. Forsythe, and J. Bienenstock. 2004. Live L. reuteri is essential for the inhibitory effect on tumor necrosis factor alpha-induced interleukin-8 expression. Infect. Immun. 72:5308-5314).

Strains of a wide variety of Lactobacillus species, including L. reuteri have been used in probiotic formulations. Lactobacillus reuteri is one of the naturally occurring inhabitants of the gastrointestinal tract of animals, and is routinely found in the intestines of healthy animals, including humans. It is known to have antimicrobial activity. See, for example, U.S. Pat. Nos. 5,439,678, 5,458,875, 5,534,253, 5,837,238, and 5,849,289. When L. reuteri cells are grown under anaerobic conditions in the presence of glycerol, they produce the antimicrobial substance known as β-hydroxy-propionaldehyde (3-HPA).

Atherosclerosis

Atherosclerotic disease and its cardiovascular consequences are the leading cause of mortality and morbidity in the United States and elsewhere. Atherosclerosis, which comes from the Greek words for “gruel” or “goo” and “hardening,” is defined as the presence of artheromas, or lesions, on the inside walls of arteries. The lesions, also known as plaque, consist of fatty deposits and other substances.

What makes atherosclerosis particularly dangerous is that it seems to have a special attraction for the large important arteries. When pieces of a plaque-filled lesion rupture from the inside wall of the arteries, the fatty material flows downstream into smaller arteries that directly supply the heart and brain, where they become stuck, preventing blood rich in nutrients and oxygen from reaching these vital organs. If total blockage occurs, the result can be a heart attack or stroke (Little, W. C., M. Constantinescu, R. J. Applegate, M. A. Kutcher, M. T. Burrows, F. R. Kahl, and W. P. Santamore. Can coronary angiography predict the site of a subsequent myocardial infarction in patients with mild-to-moderate coronary artery disease? Circulation. 1988. 78:1157-66). Traditionally, atherosclerosis has been considered a lipid metabolism disorder. The risk factors associated with atherosclerosis include high blood levels of LDL, homocysteine, hypertension, cigarette smoking, obesity and diabetes. The treatment has been focused on modulating cholesterol levels, for instance increasing the bile salt metabolism by certain lactic acid bacteria.

When evaluating the potential of using lactic acid bacteria (LAB) as effective probiotics, many consider it to be necessary to evaluate their ability of LAB to resist the effects of bile acids. Bile acids are synthesized in the liver from cholesterol and are secreted from the gall bladder into the duodenum conjugated to glycine or taurine. Their function is to emulsify dietary lipids. The most common primary bile acids in humans are cholic and chenodeoxycholic acids, which are the main end products from the cholesterol metabolism in the liver. As a result of microbial activity in the intestine, these acids then undergo chemical modifications such as deconjugation and dehydroxylation, where the amino acids hydrolyze from the conjugated form (Cardona, M. E., V. de Vanay, T. Midtvedt, and K. E. Norin. Probiotics in gnotobiotic mice. Conversion of cholesterol to coprostanol in vitro and in vivo and bile acid deconjugation in vitro. Microb Ecol Health Dis. 2000. 12:219-224; Dunne, C., L. O'Mahony, L. Murphy, G. Thornton, D. Morrissey, S. O'Halloran, M. Feeney, S. Flynn, G. Fitzgerald, D. Daly, B. Kiely, G. C. O'Sullivan, F. Shanahan, and J. K. Collins. In vitro selection criteria for probiotic bacteria of human origin: correlation with in vivo findings. Am J Clin Nutr. 2001. 73 (suppl): 386S-392S). Some gastro-intestinal (GI) bacteria, e.g. Enterococcus, Bifidobacterium, and Lactobacillus express the enzyme bile salt hydrolase (BSH), that catalyzes the hydrolysis of conjugated bile acids, which results in free glycine or taurine and unconjugated bile acid molecules (Tanaka, H., K. Doesburg, T. Iwasaki, and I. Mierau. Screening of lactic acid bacteria for bile salt hydrolase activity. J Dairy Sci. 1999. 82: 2530-2535; Bateup, J. M., M. A. McConnell, H. F. Jenkinson, and G. W. Tannock. Comparison of Lactobacillus strains with respect to bile salt hydrolase activity, colonization of the gastrointestinal tract, and growth rate of the murine host. Appl Environ Microbiol. 1995. 61(3): 1147-1149; Tannock, G. W., J. M. Bateup, and H. F. Jenkinson. Effect of sodium taurocholate on the in vitro growth of lactobacilli. Microb Ecol. 1997. 33: 163-167).

There are two main hypotheses on how the BSH expression affects the bacterial function in the GI tract. One is that some bacteria deconjugate bile salts to use the amino acid taurine as an electron acceptor, whereas the other states that the enzyme decreases the bile salt toxicity by deconjugation, since the deconjugated forms are less soluble with decreased detergent activity, thereby protecting the bacteria. Both conjugated and deconjugated bile acids have been found to exhibit antibacterial activity towards Escherichia coli, Klebsiella sp and Enterococcus sp in vitro, where the deconjugated forms have been more growth inhibitory (Dunne, C., L. O'Mahony, L. Murphy, G. Thornton, D. Morrissey, S. O'Halloran, M. Feeney, S. Flynn, G. Fitzgerald, C. Daly, B. Kiely, G. C. O'Sullivan, F. Shanahan, and J. K. Collins. In vitro selection criteria for probiotic bacteria of human origin: correlation with in vivo findings. Am J Clin Nutr. 2001. 73 (suppl): 386S-392S; Moser, S. A. and D. C. Savage. Bile salt hydrolase activity and resistance to toxicity of conjugated bile salts are unrelated properties in lactobacilli. Appl Environ Microbiol. 2001. 67 (8): 3476-3480).

The potential cholesterol lowering effects of fermented dairy products can be explained by cholesterol binding with bile acids and inhibition of micelle formation. A mechanism through which probiotic bacteria in these products may have a hypocholesterolemic effect is via bile acids, cholic and deoxycholic acids, produced from cholesterol by hepatocytes. These are conjugated with glycine and taurine, and enter the small bowel, where they are absorbed and directed to the liver. During reabsorption, the conjugated bile acids are exposed to the microflora in the intestine. Bacteria in fermented foods, e.g., lactobacilli and streptococci, hydrolyze conjugated bile acids. It is possible that a Lactobacillus strain with a high bile salt hydrolase activity in the intestine could increase the bile hydrolysis. This would lead to a faster cholesterol conversion rate to produce more bile acids. In vivo, the cholesterol decrease is due to the bile acid excretion through the feces, since deconjugated bile acids are not reabsorbed in the colon. This leads to an increase in de novo bile synthesis to keep the body's bile pool constant (St-Onge M-P., E. R. Farnworth, and P. J. H. Jones. 2000. Consumption of fermented and nonfermented dairy products: effects on cholesterol concentrations and metabolism. Am J Clin Nutr. 71: 674-681).

The deconjugation of bile acids will lower plasma cholesterol levels. However, these compounds may be further converted to secondary bile acids in the large bowel by anaerobic bacteria and secondary bile acids have been implicated as possible inducers of colon cancer. Secondary bile acids are toxic to cell lines and it is thought they exert a cytotoxic effect on colonic mucosa leading to increased cell proliferation. These hyperproliferative cells have enhanced susceptibility to mutagenic substances and, thereby increase the risk of colon cancer (Hepner, G., R. Fried, S. St. Jeor, L. Fusetti, and R. Morin. 1979. Hypercholesterolemic effect of yoghurt and milk. Am. J. Clin. Nutr. 32:19-24). Fortunately, lactic acid bacteria appear to decrease the solubility of deconjugated bile salts and secondary bile salts, thereby decreasing their bioavailability. Studies by De Boever et al. (2000) showed that L. reuteri decreased bile salt toxicity in the bacterial cultures. More importantly, addition of L. reuteri resulted in nearly complete resistance to lysis of red blood cells and inhibited the toxic effect of bile salts on HeLa cells (De Boever, P., R. Wouters, L. Verschaeve, P. Berckmans, G. Schoeters, and W. Verstraete. Protective effect of the bile salt hydrolase-active Lactobacillus reuteri against bile salt cytotoxicity. Appl Microbiol Biotechnol. 2000. 53(6):709-14).

Atherosclerosis an Immunologic Disease

Scientists are depicting a novel scheme for atherosclerosis development, suggesting that this pathology might result from an imbalance between pro-inflammatory T-cells and calming ones, the TR. This is one of the intriguing scientific results that emerge from the Second European Vascular Genomics Network Conference (EVGN Conference—Hamburg, Sep. 27th-30th 2005). These results provide new insights into the role of inflammation in heart disease and have led to development of new informative models of blood clot formation and the processes that lead to heart attacks.

Atherosclerosis starts with the formation of fatty streaks in the endothelium, as the fats in the LDL particles irritate the endothelial cells, and involves the cellular infiltration of several cell types, including monocytes and T lymphocytes. Monocytes interact with the endothelial layer, attach firmly to the endothelium, and migrate into the subendothelial space, where the monocytes differentiate into macrophages. Macrophages release a variety of chemicals, including cytokines. Production of growth factors is stimulated, which leads to cell proliferation and matrix production, as well as metalloproteinases, which leads to matrix degeneration. Thus, macrophages contribute to lesion growth and may contribute to instability and thrombotic events (Ross R. Atherosclerosis—An inflammatory disease. N Engl J Med. 1999. 340: 115-26). T-lymphocytes, have been shown to be present at all stages of atherosclerosis. Their presence provides further evidence of a connection to the immune response (Kol, A. and P. Libby. 1998. The mechanisms by which infectious agents may contribute to atherosclerosis and its clinical manifestations. Trends Cardiovasc Med. 8: 191-99; Andreotti, F., F. Burzotta, A. Mazza, A. Manzoli, K. Robinson, and A. Maseri. 1999. Homocysteine and arterial occlusive disease: a concise review. Cardiologia. 44:341-5).

The start signal of the production of inflammatory substances depends on the involvement of receptors called toll-like receptors that recognize some endogenous molecules activating the inflammatory signalling pathways (K. Edfeldt, J. Swedenborg, G. K. Hansson, and Z. Yan. 2002. Expression of Toll-Like Receptors in Human Atherosclerotic Lesions: A Possible Pathway for Plaque Activation Circulation. 105: 1158-1161).

Toll-like receptors (TLRs) recognize microbial motifs and activate a set of genes that lead to cytokine production. Traditionally, TLRs have been regarded as sensors of microbial infections, and their role is to induce an inflammatory response. However, the motifs recognized by TLRs are not unique to pathogens but are general motifs shared by entire classes of microorganisms, and its not fully understood how the immune system differentiates between commensal and pathogenic bacteria via the TLRs. Recently, data have shown that TLRs, despite their role in induction of the inflammatory response, also play a role in maintaining intestinal homeostasis by recognizing the commensal microflora (Rakoff-Nahoum, S., J. Paglino, F. Eslami-Varzaneh, S. Edberg and R. Medzhitov. 2004. Recognition of commensal microflora by toll-like receptors is required for intestinal homeostasis. Cell. 23; 118(2):229-41).

It is established that serum markers of inflammation are independent risk factors for cardiovascular morbidity and mortality. Inflammatory markers that have been associated with cardiovascular end points include pro-inflammatory cytokines such as IL-6 and TNF-a, fibrinogen, and C-reactive protein (CRP) (Libby, P., P. M. Ridker, and A. Maseri. 2001. Inflammation and atherosclerosis. Circulation. 2002.105:1135-1143; Ridker, P. M. High sensitivity C-reactive protein: potential adjunct for global risk assessment in the primary prevention of cardiovascular disease. Circulation. 103: 1813-1818).

The Role of C. pneumonie and H. pylori in Atherosclerosis

Accumulating evidence suggests that atherosclerosis is an inflammatory disease. Therefore, a great deal of attention has recently been focused on the possibility that infectious agents play a role in the etiology of cardiovascular diseases. Certain infectious agents have been implicated based on their isolation from the atheromatous plaques or on the presence of positive serology findings for organisms such as Chlamydia pneumoniae, Helicobacter pylori, herpes simplex virus, and cytomegalovirus.

Even though prospective studies have fallen short of providing definitive evidence, C. pneumoniae appears to exhibit the strongest association with atherosclerosis. C. pneumoniae has been isolated from autopsy and arthrectomy specimens and in both early and well-developed lesions. When studied by means of immunologic cytochemistry and tissue staining, the association has been found in 70-100% of cases. Possible mechanisms by which infectious agents exert their effect may include (i) local effects on the endothelium, smooth muscle cells, or macrophages or (ii) systemic effects by generating cytokines, stimulating monocytes, and promoting hypercoagulability.

Conventional Treatment for Lowering Cholesterol Levels

It has been recognized for many years that elevated serum cholesterol concentration is a risk factor associated with atherosclerosis and coronary heart disease, the latter being a major cause of death in Western countries (Barr, D. P., A. M. Russ, and H. A. Eder. 1951. Protein-lipid relationship in human plasma. II. In atherosclerosis and related conditions. Am. J. Med. 11:480-493). Numerous drugs that lower cholesterol, including the 3-hydroxy-methylglutaryl coenzyme A reductase inhibitors and drugs that increase the net excretion of bile acids, have been used to treat hypocholesterolemic (HC) individuals (Suckling, K. E., G. M. Benson, B. Bond, A. Gee, A. Glen, C. Haynes, and B. Jackson. 1991. Cholesterol lowering and bile acid excretion in the hamster with cholestyramine treatment. Atherosclerosis 89:183-190).

However, undesirable side effects of these compounds have caused concerns about their therapeutic use (Erkelens, D. W., M. G. A. Baggen, J. J. Van Doormeal, M. Kettner, J. C. Koningsberger, and M. J. T. M. Mol. 1988. Clinical experience with simvastatin compared with cholestyramine. Drugs 39(Suppl.):87-90).

Lactic Acid Bacterial as Treatment for Lowering Cholesterol Levels

In addition to these therapeutic resources, the ingestion of probiotic lactic acid bacteria possibly is a more natural method to decrease serum cholesterol concentrations in humans. Several studies report a decrease in serum cholesterol during the consumption of large doses (680 to 5000 ml/d) of fermented dairy products, but those results cannot be extrapolated to more realistic conditions of consumption (Mann, G. V. 1977. A factor in yogurt which lowers cholesterolemia in man. Atherosclerosis 26:335-340; McNamara, D. J., A. M. Lowell, and J. E. Sabb. 1989. Effect of yogurt intake on plasma lipid and lipoprotein levels in normolipidemic males. Atherosclerosis 79:167-171).

Massey showed that initially, yogurt consumption significantly reduced cholesterol by 10 to 12% in human adult males, but 2 weeks later, concentrations returned to the control values even with continued yogurt consumption (Massey, L. 1984. Effect of changing milk and yoghurt consumption on human nutrient intake and serum lipoprotein. J. Dairy Sci. 67:255-262). Similar conflicting results were obtained with experimental animals that were fed with milk and its fermented products (Hepner, G., R. S. T. Fried, S. Jeor, L. Fusetti, and R. Morin. 1979. Hypocholesterolemic effect of yogurt and milk. Am. J. Clin. Nutr. 32:19-24); Rao, D. R., C. B. Chawan, and S. R. Pulusani. 1981. Influence of milk and thermophilus milk on plasma cholesterol levels and hepatic cholesterogenesis in rats. J. Food Sci. 46:1339-1341). Rao reported a HC effect in rats fed milk that had been fermented by Streptococcus thermophilus (Rao, D. R., C. B. Chawan, and S. R. Pulusani. 1981. Influence of milk and thermophilus milk on plasma cholesterol levels and hepatic cholesterogenesis in rats. J. Food Sci. 46:1339-1341). Rodas found a similar effect in HC pigs that were fed with Lactobacillus acidophilus (Rodas, B. Z., S. E. Gilliland, and C. V. Maxwell. 1996. Hypocholesterolemic action of Lactobacillus acidophilus ATCC 43121 and calcium in swine with hypercholesterolemia induced by diet. J. Dairy Sci. 79:2121-2128).

In a study investigating the effect of L. reuteri CRL 1098 on total cholesterol, triglycerides, and the ratio of high density lipoproteins (HDL) to low density lipoproteins (LDL) in the serum of mice previously fed with a diet that had been enriched with fat, L. reuteri caused a 40% reduction in triglycerides and a 20% increase in the ratio of high density lipoprotein to low density lipoprotein without bacterial translocation of the native microflora into the spleen and liver (Taranto, M. P., F. Sesma, A. P. Ruiz Holgado, and G. F. Valdez. 1997. Bile salts hydrolase plays a key role on cholesterol removal by Lactobacillus reuteri. Biotechnol. Lett. 9:245-247). These data suggest that L. reuteri CRL 1098 is an effective hypocholesterolemic adjuvant at a low cell concentration for mice. But unlike the disclosure of the invention herein, the decrease in cholesterol was only due to BSH-activity not due to a combination of BSH-activity and immunoregulatory effects.

Lactic Acid Bacteria as Treatment for Lowering Cholesterol Levels, the Immunoregulatory Way

U.S. Patent Application No. 20050169901 relates to methods of regulating cytokine levels or activity, for diagnosis, prevention and treatment of cardiovascular disorders. The regulation of the cytokine is a switch from a Th2 to a Th1 cytokine profile in contrast to the invention herein where the switch is preferentially away from a Th1 cytokine profile towards a decrease in TNF-α production. As a probiotic the applicants mention several different bacterial genera and strains, in contrast to the invention herein where the probiotic is a specific lactic acid bacterial strain selected to be effective in decreasing TNF-α levels and simultaneously increasing the BSH-activity.

Bukowska showed that in hypercholesterolemic patients, supplementation with the probiotic bacteria Lactobacillus plantarum 299v significantly lowers concentrations of LDL cholesterol and fibrinogen (Bukowska H., J. Pieczul-Mróz, M. Jastrzêbska, K. Chelstowski, and M. Naruszewicz. 1997. Decrease in fibrinogen and LDL-cholesterol levels upon supplementation of diet with Lactobacillus plantarum in subjects with moderately elevated cholesterol. Atherosclerosis. 137:437-8). This is also described in U.S. Pat. No. 6,214,336. The same group showed that supplementation of the diet with L. plantarum may contribute to the prevention and treatment of metabolic disorders in smokers. This positive effect is thought to be directly associated with the production of propionic acid by the bacterial fermentation of fiber. They suggest that propionic acid exerts a specific antiinflammatory action through a hitherto unknown mechanism, perhaps related to the activation by ibuprofen of peroxisome proliferator-activated receptor, which modulates the nuclear transcription factor B and reduces the production of inflammatory cytokines by monocytes-macrophages (M. Naruszewicz, M-L Johansson, D. Zapolska-Downar, and H. Bukowska, Effect of Lactobacillus plantarum 299v on cardiovascular disease risk factors in smokers. Am. J. Clinical Nutrition. 2002. 76:1249-1255).

In contrast to the invention herein the abovementioned references do not describe strains capable of both increasing the BSH-activity and at the same time decreasing TNF-α levels.

As mentioned before, it has been well known for many years that elevated BSH-activity lowers serum cholesterol levels and consequently decreases the risk of atherosclerosis. It has also previously been demonstrated that atherosclerosis is an inflammatory disease and regulation of different cytokines has been suggested to halt the disease. On account of these findings, nonpathogenic bacterial strains were selected for both BSH-lowering and immunoregulatory properties. Surprisingly, some of the strains that bring about an increase in BSH-activity were simultaneously found to decrease the pro-inflammatory cytokine TNF-α levels, FIG. 1. The invention consequently refers to the use of for example L. reuteri ATCC-PTA4659, L. reuteri ATCC-6475 or L. coryniformis ATCC-PTA4660 for the manufacture of a product for the prophylaxis and/or treatment of atherosclerosis and other cardiovascular diseases, and other strains selected the same way.

It is therefore an object of this invention to provide strains of lactic acid bacteria selected for their ability of lowering serum LDL-cholesterol and decreasing the pro-inflammatory cytokine TNF-α levels. Other objects and advantages will be more fully apparent from the following disclosure and appended claims.

### BRIEF DESCRIPTION OF THE DRAWINGS

FIG. 1 is a bar graph showing the effect of Lactobacillus-conditioned media on TNF-α production by LPS-activated monocytes. Strains and controls were incubated 24 hours.

### SUMMARY OF THE INVENTION

The invention herein provides certain strains of lactic acid bacteria selected for their capability of increasing the BSH-activity and consequently lowering serum LDL-cholesterol, and simultaneously decreasing the pro-inflammatory cytokine TNF-α levels, for prophylaxis and/or treatment of atherosclerosis and other cardiovascular diseases, a method of selecting such strains, and products containing such strains.

Other objects and features of the inventions will be more fully apparent from the following disclosure and appended claims.

### DETAILED DESCRIPTION OF THE INVENTION AND PREFERRED EMBODIMENTS THEREOF

The present invention herein comprises strains of lactic acid bacteria which have been selected for their capability of reducing inflammation and increasing BSH-activity, such as in atherosclerosis. Such strains include Lactobacillus reuteri ATCC-PTA4659, which has been deposited at the American Type Culture Collection, 10801 University Blvd, Manassas, Va., on Sep. 11, 2002 under the Budapest Treaty. Lactobacillus reuteri ATCC-PTA6475 was deposited at the ATCC on Dec. 21, 2004. All restrictions to availability to the public of these strains will be irrevocably removed upon the granting of the patent. Products such as foods, nutritional additives and formulations, pharmaceuticals or medical devices containing whole cells or components derived from these strains, such as components having this capability that are present in a cell-free culture of these strains, may be formulated as is known in the art, for example a hard gelatin capsule with freeze dried culture of the Lactobacillus-strain, or its derived component.

The strains selected in example 3, for example L. reuteri ATCC PTA-6475 was added to a standard yogurt. L. reuteri ATCC PTA-6475 strain was grown and lyophilized, using standard methods for growing Lactobacillus in the dairy industry. This culture was then added to previously fermented milk, using traditional yogurt cultures, at a level of 10E+6 CFU/gram of yogurt, and the yogurt was used by humans as a prevention of atherosclerosis. Other ingestible support materials other than yoghurt can be e.g. milk, curd, fermented milks, milk based fermented products, fermented cereal based products, milk based powders.

Model systems using the appropriate cytokines are used to determine factors that reduce or increase inflammation. In the invention provided herein, an assay based on human cells is used.

THP-1 cells are a human monocytic cell line derived from leukemia patient and which are maintained at the American Type Culture Collection (ATCC No. TIB202). The origin of these cells from a human host makes them particularly relevant to study interactions of the human gastro-intestinal immune system with human commensal bacteria.

Data in this invention indicate a powerful inhibition of TNF-α production by the specific strains L. reuteri ATCC PTA-4659 and L. reuteri ATCC PTA-6475 and that this regulation is mediated by a substance released into the growth medium by these two specific strains during late log/stationary growth phase. On the contrary, two other strains of L. reuteri, were not only unable to inhibit the inflammatory response of the cells to E. coli toxin, but also induced an inflammatory response themselves.

A direct plate method with selective de Man, Rogosa and Sharpe (MRS) solid medium, containing bile salts, was used for screening strains excreting bile salt hydrolase and determine the enzymes specific activity for its substrate. Growth of BSH producing bacteria gives rise to hydrolysis and medium acidification. The hydrolysis occurs as halo formation of precipitated free bile salts around colonies (Dashkevicz, M. P. and S. D. Feighner. 1989. Development of a differential medium for bile salt hydrolase-active Lactobacillus spp. Appl Environ Microbiol. 55 (1): 11-16).

The features of the present invention will be more clearly understood by reference to the following examples, which are not to be construed as limiting the invention.

### EXAMPLE 1

### Evaluation of Strains Having the Capability of Decreasing TNF-α Levels

THP-1 cells were incubated together with either control media or conditioned media (L-CM) from the growth of selected L. reuteri strains, L. reuteri ATCC PTA-4659, L. reuteri ATCC PTA-4975, L. reuteri ATCC 55730 and L. reuteri strain PTA-4965. The conditioned media (L-CM) are cell-free supernatants from 9-hour or 24-hour cultures of each of the L. reuteri cultures. THP-1 cells were stimulated with either control medium or E. coli-derived LPS (which leads to the generation of TNFα in a normal inflammatory response) during a 3.5 hour incubation after which the cells were removed and the supernatants assayed for TNFα levels using an ELISA technique.

Materials:

THP-1 leukemic monocytic cell line (ATCC, cat number TIB202)

RPMI 1640 Medium (Gibco-Invitrogen)

Fetal Bovine Serum (Gibco-Invitrogen)

Penicillin-Streptomycin solution (Sigma)

E. coli Serotype 0127:B8 Lipopolysaccharide (Sigma, catalog number L3137)

TNF-alph/TNF-SFII human DuoSet ELISA Development Kit (R&D Systems, catalog number DY210)

Human IL-10 DuoSet, 2nd Generation-Kit (R&D Systems, catalog number DY217)

Method:

The THP-1 monocytic cell line is used. 5% (v/v) of MRS media and 5% (v/v) of Lactobacillus conditioned medium are added into the appropriate wells. Lactobacillus conditioned medium is supernatant from a 24-hour culture of Lactobacillus species in MRS media. The conditioned medium is then pH-adjusted by speed-vacuum drying and the pellet resuspended in equal volume of culture medium. Although the humidified chamber is designed to minimize liquid evaporation, after 48 hours of incubation, the cell suspension volume in the 24-well plates is reduced to about 475 μl.

100 ng/ml of E. coli serotype O127:B8 lipopolysaccharide is added into the appropriate wells, which are incubated in a 37° C., humidified, 5% CO₂ chamber. After 3.5 hours of incubation, cultures are collected into 1.5 ml centrifuge tubes and centrifuged at 1500 RCF for 5 minutes in 4° C. Supernatants are collected.

Cytokine expression is tested by ELISA (Quantikine TNF-alph/TNF-SFII human DuoSet).

The culture medium used was 10% FBS, 2% Penicillin-Streptomycin in RPMI 1640.

Results—Example 1

Addition of LPS to the THP-1 cells in the absence of L-CM led to the generation of 130 pg/ml TNFα during the 3.5 hour incubation period. This is the expected inflammatory response of the THP-1 cells to the toxin. Addition of the growth medium (MRS), which acts as a control for the L-CM additions, led to the generation of 132 pg/ml TNFα and thus MRS did not interfere with the response to LPS. The addition of 24-hour L-CM from L. reuteri ATCC PTA 4659 or L. reuteri ATCC PTA 6475 dramatically reduced the levels of LPS stimulated TNFα to only 13 and 11 pg/ml, respectively. This represents an inhibition of LPS-stimulated TNFα production of 90 and 93%, respectively.

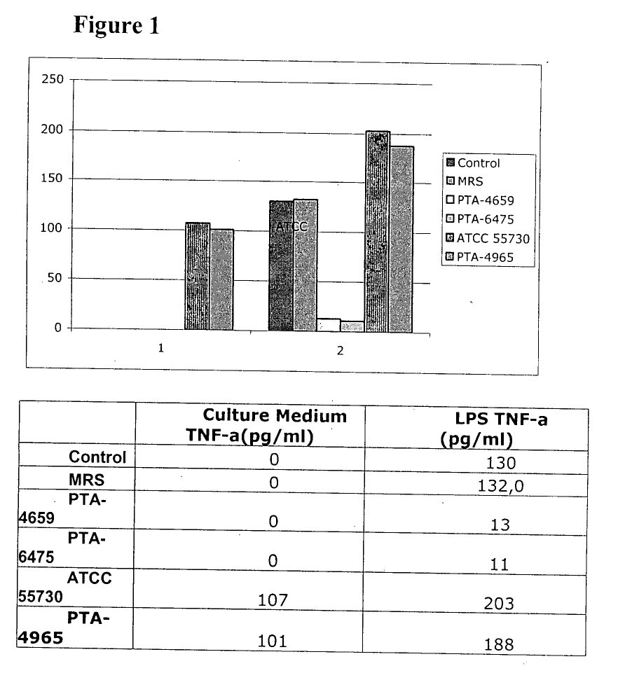

On the contrary, in the presence of 24-hour L-CM from L. reuteri ATCC 55730 and L. reuteri strain PTA-4965, LPS was still able to induce a significant rise in TNF-α compared to the levels in the absence of LPS. LPS-stimulated TNF-α production increased by 54% and 42% despite the presence of L-CM from L. reuteri ATCC 55730 and L. reuteri strain ATCC PTA-4965, respectively (FIG. 1).

Similar experiments performed with 9-hour L-CM from L. reuteri ATCC PTA 4659 or L. reuteri ATCC PTA 6475 demonstrated that the inhibitory effect on LPS-stimulated TNFα production was considerably less but still there. Thus, longer incubations of the L. reuteri strains, with harvesting of the L-CM in late log/stationary phase of growth, leads to improved efficacy in inhibiting TNF-α production.

### EXAMPLE 2

### Direct Plate Assay—Evaluation of Strains with Extracellular BSH Activity

Strains of human lactic acid bacteria were grown in oxygen limited conditions at 37° C. in MRS broth (Acumedia Manufacturers, Inc. Baltimore, Md.) overnight, and inoculated in lactobacilli carrying medium (LCM) with 10% glycerol (BDH Laboratory Supplies, England).

| | GDCA | | TDCA | |
|---|---|---|---|---|
| Strain | activity | growth | activity | growth |
| Lactobacillus reuteri MV10-1a | − | + | − | + |
| Lactobacillus reuteri ATCC 55730 | + | + | − | + |
| Lactobacillus reuteri MM2-2 | + | + | + | + |
| Lactobacillus reuteri MF52-1F | + | + | + | + |
| Lactobacillus reuteri DSM20016 | + | + | + | + |
| Lactobacillus rhamnosus MV45-2a | + | + | + | + |
| Lactobacillus gasseri MV7-2a | + | + | + | + |
| Lactobacillus gasseri MV1-21g | − | + | − | + |
| Lactobacillus reuteri ATCC PTA- | − | − | + | + |
| 4965 | | | | |
| Lactobacillus paracasei MV49-2b | + | + | + | + |
| Lactobacillus reuteri ATCC PTA- | + | + | + | + |
| 4659 | | | | |
| Lactobacillus reuteri ATCC PTA- | + | + | + | + |
| 6475 | | | | |
| Lactobacillus reuteri FJ3 | + | + | + | + |
| Lactobacillus reuteri MM4-2a | + | + | + | + |
| Lactobacillus reuteri FJ1 | + | + | + | + |
| Lactobacillus rhamnosus GG | − | + | − | + |
| Lactobacillus coryniformis MM7 | + | + | − | + |
| Streptococcus salivarius subsp | + | + | − | + |
| thermophilus | | | | |
| Lactobacillus delbrueckii subsp | − | − | − | + |
| bulgaricus | | | | |
| Lactobacillus casei shirota | + | + | − | + |
| Lactobacillus fermentum Kx356C2 | + | + | + | + |
| Lactobacillus brevis ATCC 14869 | + | + | − | + |
| Lactobacillus fermentum Kx338A2 | − | − | − | + |
| Lactobacillus plantarum 299v | + | + | − | + |
| Lactobacillus gasseri Kx338A3 | − | − | − | + |
| Lactobacillus gasseri Kx315A1 | + | + | + | + |

The stock cultures were stored at −80° C. for further use. The strains were obtained from the BioGaia AB laboratories and strain collection in Lund (Sweden), Raleigh (NC, United States of America) and Lantbruksuniversitetet (University of Agriculture), Uppsala (Sweden).

To screen for extracellular BSH activity, the strains were streaked from overnight cultures on MRS-cysteine (MRS-c) agar (Acumedia) plates containing 3 mM of the bile salts, GDCA (Sigma, Steinheim, Germany), TDCA (Sigma), GCA (Sigma), and TCA (Fluka, Sigma-Aldrich, Germany), respectively. The plates were incubated anaerobically (AnaeroGen, Oxoid, UK) for 48 hours at 37° C. The precipitation, which is the result of bile acid deconjugation, was measured visually, and thereby subjectively, hence the activity is mentioned no activity (−) or activity (+). MRS-c agar plates with no added bile salt were used as growth and negative controls.

### EXAMPLE 3

### Selection of Strains with BSH-Activity and Capability to Simultaneously Decrease TNF-α Levels

| | TNF-a | BSH- | |
|---|---|---|---|
| Strain | reduction | activity | Selection |
| L. reuteri ATCC | + + | + + | S |
| PTA-4659 | | | |
| L. reuteri ATCC | + + | + + | S |
| PTA-6475 | | | |
| L. reuteri ATCC | − − | + − | — |
| 55730 | | | |
| L. reuteri ATCC | − − | − + | — |
| PTA-4965 | | | |

The data in the above table confirm the surprising finding that the different strains of L. reuteri have varying effects on TNF-α and BSH production, and that strains L. reuteri ATCC PTA-4659 and L. reuteri ATCC PTA-6475 are particularly suitable for use in atherosclerosis.

### EXAMPLE 4

### Use of the Conditioned Medium

Using the method in example 1, the conditioned medium from one effectively TNF-α decreasing strain was selected, in this example the medium from L. reuteri ATCC PTA-4659. This medium was produced in larger scale by growing the strain in de Man, Rogosa, Sharpe (MRS) (Difco, Sparks, Md.). Overnight cultures of lactobacilli were diluted to an OD₆₀₀ of 1.0 (representing approximately 10⁹ cells/ml) and further diluted 1:10 and grown for an additional 24 h. Bacterial cell-free conditioned medium was collected by centrifugation at 8500 rpm for 10 min at 4° C. Conditioned medium was separated from the cell pellet and then filtered through a 0.22 μm pore filter unit (Millipore, Bedford, Mass.). The conditioned medium was then lyophilized and formulated, using standard methods, to make a tablet. This tablet was used as a drug by humans to effectively treat atherosclerosis.

### EXAMPLE 5

### Use of Selected Anti-Inflammatory Lactobacillus reuteri Strains

Using the methods in example 1 and 2 one strain effectively decreasing TNF-α and at the same time increasing BSH-activity was selected, in this experiment L. reuteri ATCC PTA-4659. The L. reuteri strain was then lyophilized and formulated, using standard methods, to make a capsule, in the range of 10⁵-10⁹ cfu. This capsule was used as a drug by humans to effectively reduce atherosclerosis.

### EXAMPLE 6

### Lactobacillus reuteri Reducing Caroteid Plaques in Atherosclerosis

A total of 1059 patients are given valid ultrasound measurements at baseline and 1-year follow up. At baseline and follow-up, the same ultrasound imaging system and transducer (Acuson Xp10 128, ART upgraded, with a 7.5-MHz linear-array transducer, aperture size 38 mm, SIEMENS) are used. The B-mode image adjustment parameters are preset to fixed values and are not changed during the course of either survey. With the subject in a supine position, head turned slightly to the left, the right carotid artery is scanned with several different angles of insonation, both longitudinally and transversely, from just above the clavicle to as far distal to the bifurcation as possible. A plaque is defined as a local protrusion of the vessel wall into the lumen of at least 50% compared with the adjacent intima-media thickness (IMT). In each subject, a maximum of 6 plaques are registered in the near and far walls of the common carotid, bifurcation, and internal carotid, respectively. For each plaque, a still image is recorded with the transducer parallel to the vessel wall and as perpendicular to the point of maximum plaque thickness as possible, with the regional expansion selection set to 38 mm×20 mm. All recordings are done on a Panasonic 7650 video player with Super VHS tape.

At baseline 1059 men have plaque present (Table 1). Carotid plaque area decreased at any age. Mean total plaque area (SE) at baseline is 24.1 mm². In the follow-up period after eating a daily dose of L. Reuteri ATCC PTA-4659 (10⁸ CFU), all the persons have a decrease in total plaque area. The mean decrease is 9.0 mm².

| | | Plaque area at | Δ Plaque area |
|---|---|---|---|
| Age | n | baseline mm2 | mm2 |
| <60 | 352 | 18.9 | 6.4 |
| 60-64 | 291 | 24.2 | 9.4 |
| 65-70 | 289 | 27.2 | 9.7 |
| >70 | 127 | 27.2 | 11.5 |
| SE | | 24.1 | 9.0 |

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A biologically pure culture of a Lactobacillus strain selected for a capability of increasing the BSH-activity and consequently lowering serum LDL-cholesterol, and simultaneously decreasing the pro-inflammatory cytokine TNF-α levels, for prophylaxis and/or treatment of atherosclerosis and other cardiovascular diseases.

2. The biologically pure culture of claim 1, wherein the Lactobacillus strain is selected from the group consisting of Lactobacillus reuteri ATCC PTA-4659 and ATCC PTA-6475.

3. A method for selecting bacterial strains effective for treating inflammation in atherosclerosis, comprising: using THP-1 monocytic cell line from a human source to identify strains that are effective in decreasing TNFα levels.

4. An atherosclerosis-associated inflammation-reducing component derived from a biologically pure culture of a strain of Lactobacillus according to claim 1, said component obtained from a cell-free culture supernatant after growth of said strain, and having the capability of reducing TNFα amount.

5. A cell-free culture supernatant isolated from a biologically pure culture of Lactobacillus reuteri strains ATCC PTA-4659 or ATCC PTA-6475.

6. A food composition comprising an ingestible support and an atherosclerosis-associated inflammation-reducing component derived from of a strain of Lactobacillus selected from the group consisting Lactobacillus reuteri strains ATCC PTA-4659 and ATCC PTA-6475.

7. The food composition of claim 6, wherein the inflammation-reducing component comprises cells of a biologically pure culture of the strain of Lactobacillus.

8. The food composition of claim 7, wherein the Lactobacillus strain is selected from the group consisting of Lactobacillus reuteri ATCC PTA-4659 and ATCC PTA-6475.

9. A pharmaceutical composition comprising a pharmaceutical carrier and an atherosclerosis associated inflammation-reducing component derived from of a strain of Lactobacillus selected from the group consisting of Lactobacillus reuteri strains ATCC PTA-4659 and ATCC PTA-6475.

10. The pharmaceutical composition of claim 10, wherein the component comprises cells of a biologically pure culture of the strain of Lactobacillus.

11. The pharmaceutical composition of claim 11, wherein the Lactobdcillus strain is selected from the group consisting of Lactobacillus reuteri ATCC PTA-4659 and ATCC PTA-6475.

12. A nutritional supplement comprising an ingestible support and an atherosclerosis associated inflammation-reducing component derived from of a strain of Lactobacillus selected from the group consisting of Lactobacillus reuteri strains ATCC PTA-4659 ATCC PTA-6475.

13. The nutritional supplement of claim 13, wherein the component comprises cells of a biologically pure culture of the strain of Lactobacillus.

14. A method for preparing a food composition, comprising:
a. selecting strains of Lactobacillus according to claim 3;
b. obtaining an anti-inflammatory component from said strains; and
c. adding said component to an ingestible support to provide a food.

15. A method for preparing a pharmaceutical composition, comprising:
a. selecting strains of Lactobacillus according to claim 3;
b. obtaining an anti-inflammatory component from said strains; and
c. adding said component to pharmaceutical carrier to provide a pharmaceutical composition.

16. A method for preparing a nutritional supplement, comprising:
a. selecting strains of Lactobacillus according to claim 3;
b. obtaining an anti-inflammatory component from said strains; and
c. adding said component to an ingestible support to provide a nutritional supplement.

17. An agent for treatment or prophylaxis of inflammation associated with atherosclerosis comprising an anti-inflammatory component from strains of Lactobacillus according to claims 1 and 3.

18. A method for treatment or prophylaxis of inflammation associated with atherosclerosis comprising selecting at least one strain of Lactobacillusi, said at least one strain characterized in that it is capable of reducing atherosclerosis, and administering cells of said at least one strain to a human.

19. The method of claim 18, wherein the cells are administered orally.
